(19) United States
(12) Patent Application Publication
(10) Pub. No.: US 2024/0279149 A1
(43) Pub. Date: Aug. 22, 2024

(54) HALOGENATED CURCUMIN DERIVATIVE, PREPARATION METHOD THEREFOR, AND APPLICATION THEREOF IN PRESERVATION OF AQUATIC PRODUCTS

(71) Applicant: ZHEJIANG OCEAN UNIVERSITY, Zhejiang (CN)

(72) Inventors: Bin ZHANG, Zhejiang (CN); Long JIAO, Zhejiang (CN); Yuexiang ZHAN, Zhejiang (CN); Yintao ZHOU, Zhejiang (CN)

(73) Assignee: ZHEJIANG OCEAN UNIVERSITY, Zhejiang (CN)

(21) Appl. No.: 18/561,292

(22) PCT Filed: Jul. 27, 2022
(86) PCT No.: PCT/CN2022/108306

§ 371 Date: Nov. 16, 2023

(30) Foreign Application Priority Data
Jul. 28, 2021 (CN) 202110858834.6

(51) Int. Cl. C07C 49/225; A23B 4/20

(52) U.S. Cl. CPC C07C 49/225; A23B 4/20

(57) ABSTRACT

The present invention relates to the field of aquatic product processing and storage; to solve the problems in that a photosensitizer generates low active oxygen during photodynamic sterilization and that the preservation effect of aquatic products needs to be improved, a halogenated curcumin derivative, a preparation method therefor, and an application thereof in the preservation of the aquatic product are disclosed. The halogenated curcumin derivative contains mono-substituted or bi-substituted halogenated groups; the halogenated curcumin derivative can be prepared by a one-step method and used for photodynamic sterilization of aquatic products. The preparation steps of the present invention are simple, the reaction conditions are mild, and the prepared halogenated curcumin derivative has high singlet oxygen yield under blue light irradiation. The present invention has good sterilization and preservation effects on aquatic products, can effectively prolong the shelf life of fresh shrimp, and has little impact on the sensory properties of the food.

### TECHNICAL FIELD

The present invention relates to the field of aquatic product processing and storage, and in particular to a halogenated curcumin derivative, a preparation method therefor, and an application thereof in the preservation of aquatic products.

### BACKGROUND

Photodynamic sterilization is a new type of non-thermal sterilization technology, and the principle thereof is to use photosensitizer molecules to sensitize oxygen under visible light irradiation to generate cytotoxic reactive oxygen species (mainly singlet oxygen species, 102) to kill pathogenic microorganisms. Due to the advantages including broad-spectrum sterilization properties, non-toxicity, and no drug resistance, the photodynamic sterilization has great application prospects in the field of food sterilization, especially in the sterilization and preservation of aquatic products. Moreover, the photodynamic sterilization technology can effectively maintain the original nutritional composition, flavor, color, taste, and freshness of the aquatic products under the premise of achieving efficient inactivation of pathogenic bacteria. At present, the photosensitizers that can be applied in the field of food sterilization generate less reactive oxygen species, resulting in poor photodynamic sterilization effect and shorter shelf life of the aquatic products. Therefore, there is an urgent need for developing a photosensitizer with more reactive oxygen species generated.

For example, a “method for photodynamic cold sterilization and preservation of sturgeon” is disclosed in Chinese Patent with a publication number of CN110150372A, and the method includes pre-treating sturgeon, taking, slicing, and cleaning meat with normal saline, evenly spraying water with photosensitizer on sturgeon slices, and then illuminating the sturgeon slices with LED light to complete the sterilization process. The invention uses food-grade curcumin as a photosensitizer. Due to the low efficiency of curcumin intersystem crossing (ISC), less reactive oxygen species are generated in the photodynamic sterilization process, and the lack of the reactive oxygen species affects its photodynamic sterilization effect.

### SUMMARY

In order to solve the problem of less reactive oxygen species generated by photosensitizers in photodynamic sterilization in the prior art and need for improving the preservation effect, a halogenated curcumin derivative, a preparation method therefor, and an application thereof in the preservation of aquatic products are disclosed. The halogenated curcumin derivative has high intersystem crossing efficiency under the influence of heavy atom effect of non-metallic halogen atoms, resulting in a high singlet oxygen yield. Preparation steps are simple, and the preservation effect of the aquatic products is good.

In order to achieve the above purposes, the present invention adopts the following technical solution:

A halogenated curcumin derivative is provided, and the halogenated curcumin derivative has the following structural formula I:

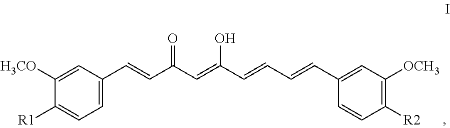

where R1 is one of F, Cl, Br, and I; and R2 is one of F, Cl, Br, I, and OH.

The halogenated curcumin derivative structure in the present invention contains non-metallic halogen heavy atoms, which can enhance the spin-orbit coupling effect, enhancing the intersystem crossing efficiency of the derivative to obtain a long life triple excited state, enabling it to obtain an enhanced singlet oxygen yield, and thereby improving its photodynamic sterilization effect.

A method for preparing the halogenated curcumin derivative is provided and includes the following steps:
(1) dissolving acetylacetone and boric anhydride in a solvent, stirring until complete dissolution, adding raw material a, raw material b, and tributyl borate, and continuing stirring until complete dissolution;
(2) stirring for reaction after adding catalyst dropwise; and
(3) after the reaction, adding a hydrochloric acid solution, separating an organic phase after stirring and extracting the organic phase with an extractant, carrying out rotary evaporation to obtain a crude product after drying, and purifying the crude product by column chromatography to obtain a target product curcumin derivative,
   where the raw material a in step (1) has the following structural formula II:

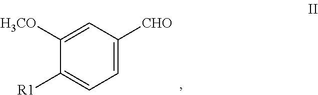

and the raw material b has the following structural formula III:

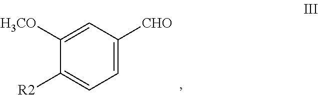

where R1 is one of F, Cl, Br, and I; and R2 is one of F, Cl, Br, I, and OH.

The present invention uses acetylacetone and methoxybenzaldehyde containing halogenated groups as raw materials, boric anhydride as a protective agent, and tributyl borate as a dehydrating agent to prepare the halogenated curcumin derivative through condensation reaction. The method for preparing the halogenated curcumin derivative has simple steps and mild reaction conditions, and the purity of the obtained product is high.

As a preferred option, a molar ratio of acetylacetone, boric anhydride, the raw material a, the raw material b, and tributyl borate in step (1) is 1:(1 to 4):(0.5 to 1.5):(0.5 to 1.5):(1 to 3); and the solvent described in step (1) is ethyl acetate.

As a preferred option, the catalyst in step (2) is piperidine or n-butylamine, a mass of the catalyst is 0.10% to 10% of acetylacetone, and the stirring reaction time is 12 h to 24 h.

As a preferred option, steps (1) and (2) are carried out under nitrogen protection conditions at 25° C. to 45° C.

As a preferred option, in step (3), a volume concentration of the hydrochloric acid solution is 10% to 30%; and a volume ratio of the hydrochloric acid solution to the solution obtained in step (1) is (0.8 to 1.2):2.

An application of the halogenated curcumin derivative in the preservation of aquatic products is provided, and the halogenated curcumin derivative is used as a photosensitizer for photodynamic sterilization of the aquatic products.

The halogenated curcumin derivative of the present invention as a photosensitizer is applied to the photodynamic sterilization and preservation of the aquatic products, because the halogenated curcumin derivative has high intersystem crossing efficiency under the influence of heavy atom effect of non-metallic halogen atoms, more reactive oxygen species are generated in the photodynamic sterilization process, the sterilization and preservation effect is good, and it can preserve the original flavor of food without affecting its taste.

As a preferred option, the photodynamic sterilization includes the following steps:
(A) preparing a halogenated curcumin derivative aqueous solution in a dark water tank;
(B) draining the surface water after cleaning the aquatic products with sterile normal saline;
(C) soaking the aquatic products prepared in step (B) in the halogenated curcumin derivative aqueous solution prepared in step (A) by adopting a soaking method, and taking out the aquatic products after soaking in the aqueous solution for 10 min; and
(D) immediately placing the aquatic products obtained in step (C) under a blue LED matrix for irradiation.

The preferred aquatic products are large yellow croaker, small yellow croaker, and Chinese tube whip shrimp. The color of the halogenated curcumin aqueous solution is yellow. Choosing a yellow colored aquatic product will not have a significant effect on its appearance.

As a preferred option, a concentration of the halogenated curcumin derivative aqueous solution in step (A) is 10 μmol/L to 40 μmol/L.

As a preferred option, an optical power of the blue LED matrix in step (D) is 5 W to 20 W, a wavelength range is 400 nm to 480 nm, and the irradiation time is 60 s to 180 s.

Therefore, the present invention has the following beneficial effects: (1) the halogenated curcumin derivative has a high singlet oxygen yield under blue light irradiation; (2) preparation steps of the halogenated curcumin derivative are simple, and reaction conditions are mild; and (3) the halogenated curcumin derivative has a good sterilization and preservation effect on the aquatic products, and can effectively prolong the shelf life of fresh shrimp and have little effect on food sensory properties.

### BRIEF DESCRIPTION OF THE DRAWINGS

FIG. 1 is a mass spectrum of the product obtained from Embodiment 1.

FIG. 2 is a UV absorption spectrum of the product obtained from Embodiment 1.

FIG. 3 is a fluorescence spectrum of the product obtained from Embodiment 1.

FIG. 4 is a comparison diagram of the singlet oxygen generation ability between the product obtained from Embodiment 1 and natural curcumin.

FIG. 5 is a schematic diagram of the flow illustrating the application of the halogenated curcumin derivative in photodynamic sterilization and preservation.

FIG. 6 is a diagram showing the change of the total number of bacteria of Chinese tube whip shrimp.

FIG. 7 is a sensory score diagram of Chinese tube whip shrimp.

### DETAILED DESCRIPTION

The present invention will be further described in conjunction with the accompanying drawings and specific embodiments.

### Embodiment 1

A brominated curcumin derivative is provided, a structural formula thereof is

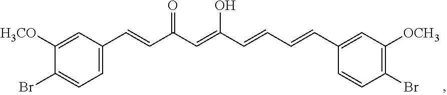 [SMILES: COc1cc(/C=C/C=C/C(O)=C/C(=O)/C=C/c2ccc(Br)c(OC)c2)ccc1Br]

and preparation steps thereof are as follows:
(1) 1 mmol of acetylacetone and equal molar amounts of boric anhydride were dissolved in 20 mL of ethyl acetate under the protection conditions of N₂ at 25° C., and stirred for 0.5 h;
(2) 2 mmol of 4-bromo-3-methoxybenzaldehyde and 2 mmol of tributyl borate were added, and continuously stirred for 0.5 h;
(3) 0.01 mmol of catalytic amount of n-butylamine was added dropwise and continuously stirred for reaction for 24 h;
(4) after the reaction was completed, 10 mL of hydrochloric acid solution with a volume concentration of 10% was added and continuously stirred for 1 h; and
(5) the organic phase was separated, extracted with ethyl acetate, and dried with anhydrous sodium sulfate, rotary evaporation was carried out to obtain a crude product, and the crude product was purified by column chromatography to obtain a target product brominated curcumin derivative.

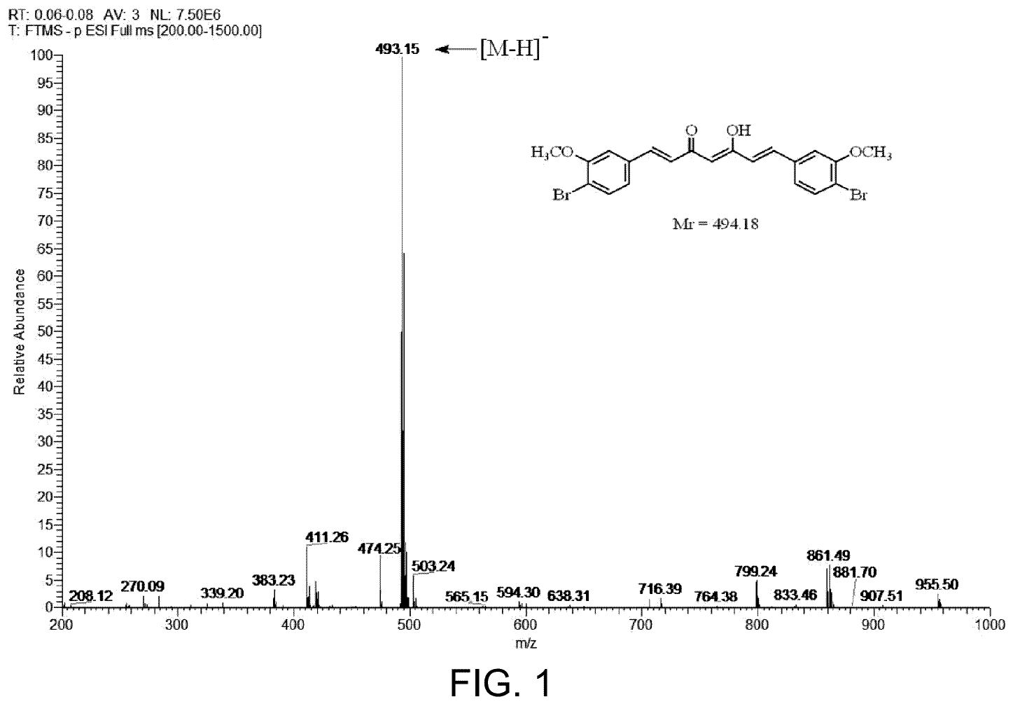

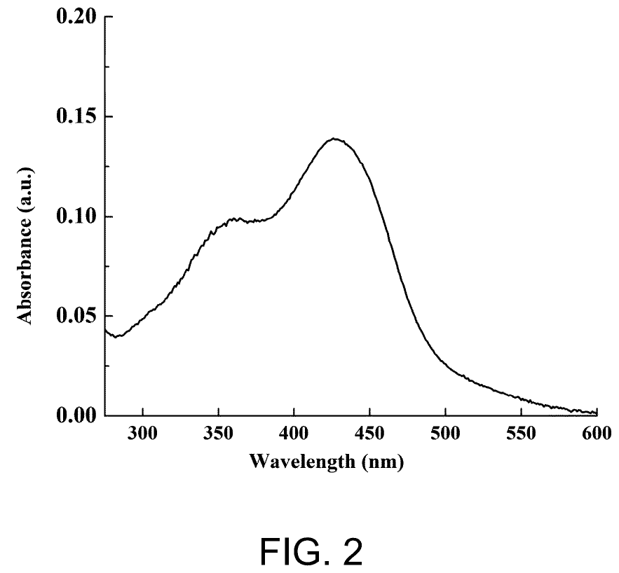

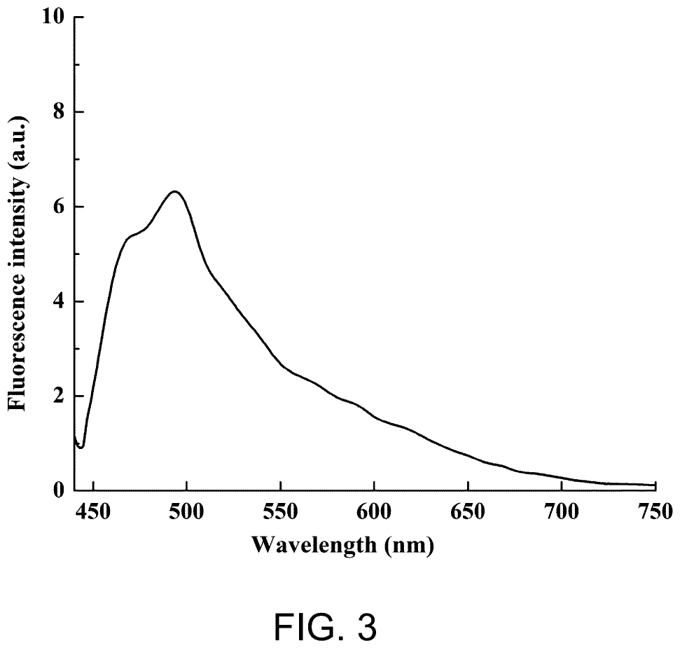

The product obtained from Embodiment 1 was detected using ESI mass spectrometry, and the detection results are shown in FIG. 1. The product obtained from Embodiment 1 was formulated to a solution with a concentration of 5 μmol/L according to theoretical calculations, the UV absorption spectrum and fluorescence spectrum of the solution were scanned, and the results are shown in FIG. 2 and FIG. 3. FIG. 1 to FIG. 3 indicate that the preparation method has successfully prepared the brominated curcumin derivative.

### Embodiment 2

A mono-brominated curcumin derivative is provided, a structural formula thereof is

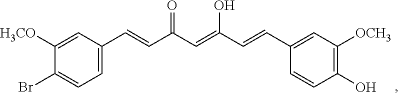 [SMILES: COc1cc(/C=C/C(O)=C/C(=O)/C=C/c2ccc(Br)c(OC)c2)ccc1O]

and preparation steps thereof are as follows:
(1) 1 mmol of acetylacetone and equal molar amounts of boric anhydride were dissolved in 20 mL of ethyl acetate under the protection conditions of N₂ at 25° C., and stirred for 0.5 h;
(2) 1 mmol of 4-bromo-3-methoxybenzaldehyde, 1 mmol of vanillic aldehyde and 2 mmol of tributyl borate were added, and continuously stirred for 0.5 h;
(3) 0.01 mmol of catalytic amount of n-butylamine was added dropwise and continuously stirred for reaction for 24 h;
(4) after the reaction was completed, 10 mL of hydrochloric acid solution with a volume concentration of 10% was added and continuously stirred for 1 h; and
(5) the organic phase was separated, extracted with ethyl acetate, and dried with anhydrous sodium sulfate, rotary evaporation was carried out to obtain a crude product, and the crude product was purified by column chromatography to obtain a target product mono-brominated curcumin derivative.

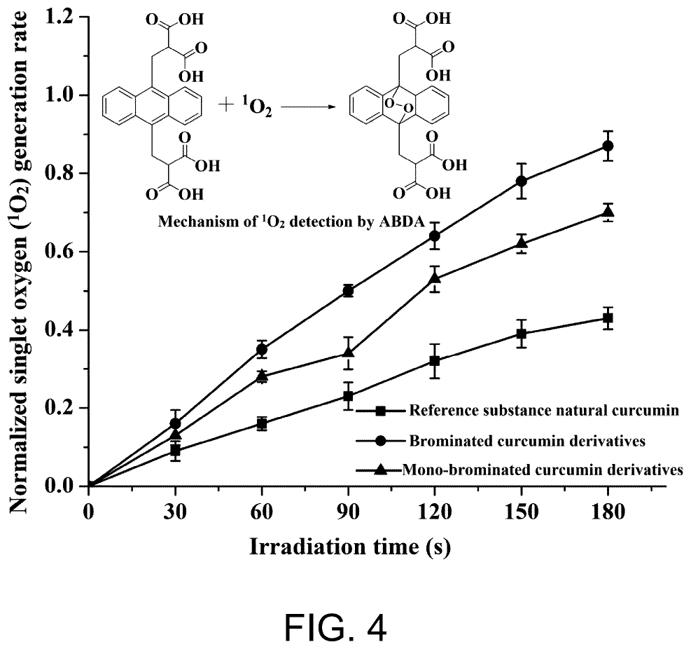

The singlet oxygen generation ability of the brominated curcumin derivative obtained from Embodiment 1 and the mono-brominated curcumin derivative obtained from Embodiment 2 was evaluated using the 9,10-anthracyl-bis(methylene)dipropionic acid (ABDA) absorbance decay method. The detection steps are as follows:
(1) the mixed brominated curcumin derivative aqueous solution containing the brominated curcumin derivative prepared from Embodiment 1 and the ABDA with the final concentrations of 5 μmol/L and 50 μmol/L, respectively, was prepared;
(2) the mixed mono-brominated curcumin derivative aqueous solution containing the mono-brominated curcumin derivative prepared from Embodiment 2 and the ABDA with the final concentrations of 5 μmol/L and 50 μmol/L, respectively, was prepared;
(3) the reference substance natural curcumin aqueous solution containing the natural curcumin and the ABDA with the final concentrations of 5 μmol/L and 50 μmol/L, respectively, was prepared; and
(4) the mixed brominated curcumin derivative aqueous solution, the mixed mono-brominated curcumin derivative aqueous solution, and the reference substance natural curcumin aqueous solution were irradiated with a blue LED matrix light source (470 nm, 20 mW/cm²) for 180 s, the absorbance value of the ABDA was recorded for every 30 s, and the absorbance decay curve was drawn. The comparison results are shown in FIG. 4.

From FIG. 4, it can be seen that the curcumin derivative substituted with bromine atoms have higher singlet oxygen generation ability than the natural curcumin Since the heavy atom effect of double bromine substitution is stronger than that of single bromine substitution, the singlet oxygen generation ability of the brominated curcumin derivative obtained in Embodiment 1 is higher than that of the single bromine substituted curcumin derivative obtained in Embodiment 2.

### Application Example 1

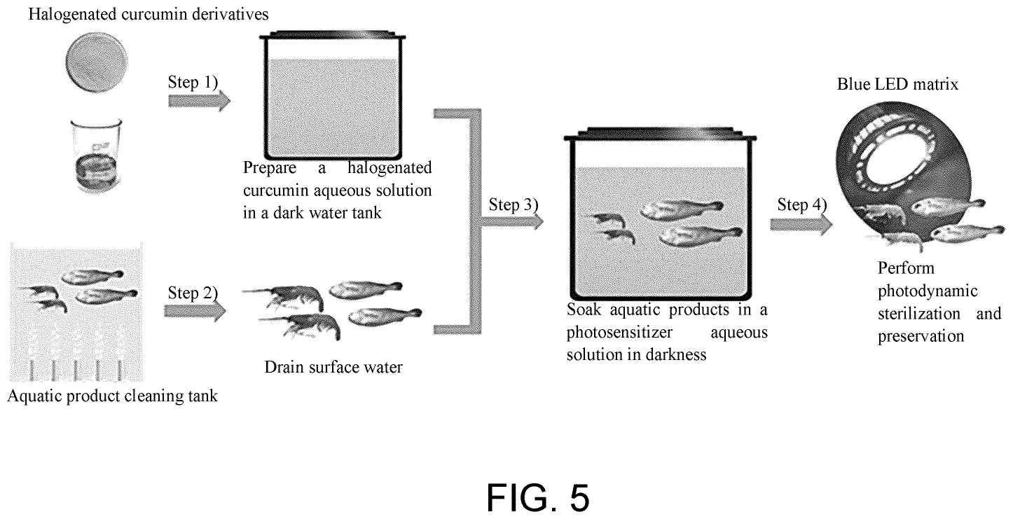

A photodynamic sterilization method for fresh shrimp is provided, and the steps are shown in FIG. 5:
(1) a brominated curcumin derivative aqueous solution obtained in Embodiment 1 was prepared in a dark water tank with a concentration of 20 μmol/L;
(2) fresh and intact Chinese tube whip shrimps with bright color and uniform size were selected, soaked in the ice water (1:2, w/v), frozen to death, washed with sterile normal saline, and drained the surface water;
(3) the aquatic product prepared in step (2) was soaked in the brominated curcumin derivative aqueous solution prepared in step (1) by adopting a soaking method, and the aquatic products were taken out after soaking in the aqueous solution for 10 min; and
(4) the aquatic products obtained in step (3) was immediately placed under a blue LED matrix with an optical power of 10 W, an irradiation time of 120 s and a wavelength of 470 nm.

### Comparative Application Example 1

A photodynamic sterilization method for fresh shrimp is provided, and the steps are as follows:
(1) a natural curcumin aqueous solution was prepared in a dark water tank with a concentration of 20 μmol/L;
(2) fresh and intact Chinese tube whip shrimps with bright color and uniform size were selected, soaked in the ice water (1:2, w/v), frozen to death, washed with sterile normal saline, and drained the surface water;
(3) the aquatic product prepared in step (2) was soaked in the natural curcumin aqueous solution prepared in step (1) by adopting a soaking method, and the aquatic products were taken out after soaking in the aqueous solution for 10 min; and
(4) the aquatic products obtained in step (3) was immediately placed under a blue LED matrix with an optical power of 10 W, a irradiation time of 120 s and a wavelength of 470 nm.

### Comparative Application Example 2

(1) Fresh and intact Chinese tube whip shrimps with bright color and uniform size were selected, soaked in the ice water (1:2, w/v), frozen to death, washed with sterile normal saline, and drained the surface water.

The total sterilization effect experiment and sensory evaluation of the Chinese tube whip shrimps obtained from Application Example 1, Comparative Application Example 1, and Comparative Application Example 2 were carried out. The specific steps are as follows:
(1) the Chinese tube whip shrimps obtained from Application Example 1, Comparative Application Example 1, and Comparative Application Example 2 were put into a fresh-keeping bag of a fresh-keeping box, and stored in a refrigerator at 4° C.;
(2) when stored for 2, 4, 6, 8, and 10 days, according to the method specified in the national standard GB/T 4789.2-2016 Food Microbiological Examination—Aerobic Plate Count, the total number of colonies of the Chinese tube whip shrimps treated in step (1) was measured; and
(3) when stored for 2, 4, 6, 8, and 10 days, a sensory evaluation team conducted sensory scores on the stored shrimps; the sensory score team was composed of 10 strictly trained personnel to evaluate the appearance, odor, and posture of the processed Chinese tube whip shrimps, and the average value was taken. The scoring criteria are shown in Table 1, and the sensory score result is the sum of the 3 indicator scores with a total score of 18 indicating absolute freshness and 9 indicating significant quality deterioration.

TABLE 1
Sensory score criteria for Chinese tube whip shrimp

| Scoring | Sensory score | | |
|---|---|---|---|
| items | 6 points | 3 points | 0 point |
| Appearance | The shrimp body has a | The shrimp has a dark | The shrimp body has a |
| | natural color without a | color with a blackhead | dark and dull color with a |
| | blackhead | | serious blackhead |
| Odor | The fresh shrimp has an | No freshness | Smelly |
| | inherent odor | | |
| Posture | The shrimp body is intact, | The shrimp body is intact, | The shrimp body is |
| | the shell does not fall off, the | there is some rupture of | basically intact, there are |
| | connecting membrane does | the connecting membrane, | ruptures of the connecting |
| | not break, and there is no | and the shell is soft and | membrane, and the shell |
| | soft-shell shrimp | does not fall off, and there | falls off, and there are soft- |
| | | is no soft-shell shrimp | shell shrimps |

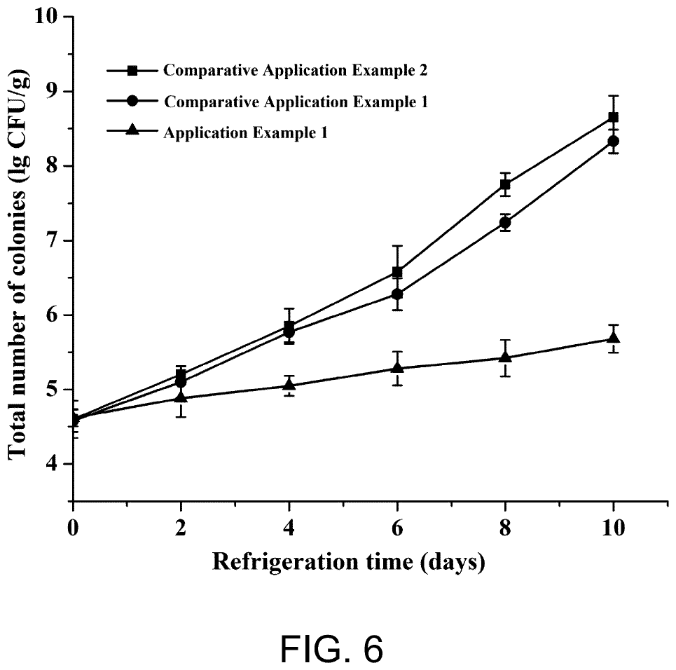

The killing effect of total number of colonies is shown in FIG. 6. It can be seen from FIG. 6 that compared with Comparative Application Example 1 and Comparative Application Example 2, the bacterial growth of Chinese tube whip shrimp photo-dynamically treated with the brominated curcumin derivative in Application Example 1 is significantly inhibited (p<0.05). Generally, the shrimp with a total number of colonies of ≤10⁵ CFU/g is considered as first-level freshness, and the shrimp with a total number of colonies of ≤5×10⁵ CFU/g is at the second-level freshness, and when the total number of bacterial colonies reaches 106 CFU/g, it indicates that the shrimp has rotted and cannot be consumed, which is the end of the shelf life. From this, it can be seen that the shelf life of refrigerated Chinese tube whip shrimp in Comparative Application Example 1 and Comparative Application Example 2 is only 4 days. The refrigerated Chinese tube whip shrimp obtained from Application Example 1 remains at first-level freshness on the 4^{th} day of refrigeration, and after 10 days of refrigeration, it still approached second-level freshness. Therefore, the brominated curcumin derivative involved in the present invention can efficiently kill the colonies of the fresh shrimp by photodynamic treatment on Chinese tube whip shrimp, and the shelf life of fresh shrimp is significantly prolonged.

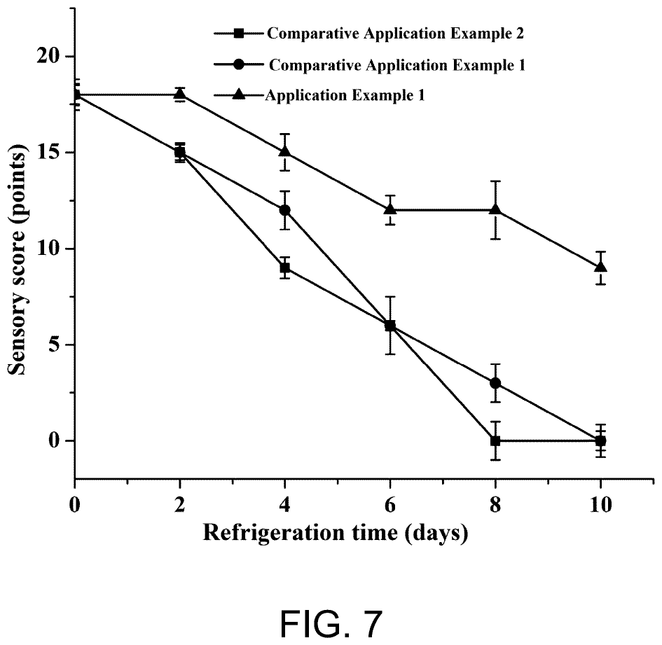

The sensory evaluation score is shown in FIG. 7. It can be seen from FIG. 7 that after photodynamic treatment with the brominated curcumin derivative, the sensory score of Chinese tube whip shrimp is consistently higher than that of Comparative Application Example 1 and Comparative Application Example 2, and there is still no significant quality deterioration after 10 days of storage. However, after 4 days of storage for Comparative Application Example 1 and Comparative Application Example 2, significant quality deterioration has been observed. It can be seen that the sensory flavor of Chinese tube whip shrimp did not significantly decrease after photodynamic treatment with the brominated curcumin derivative, indicating that the photodynamic treatment of the brominated curcumin derivative can effectively prolong the shelf life of Chinese tube whip shrimp.

1. A halogenated curcumin derivative, having the following structural formula I: 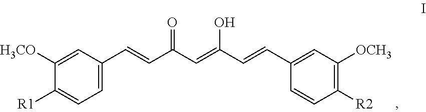
wherein R1 is one of F, Cl, Br, and I; and R2 is one of F, Cl, Br, I, and OH.

2. A method for preparing the halogenated curcumin derivative according to claim 1, comprising the following steps:
step (1) dissolving acetylacetone and boric anhydride in a solvent, stirring until complete dissolution, adding a raw material a, a raw material b, and tributyl borate, and continuing stirring until complete dissolution;
step (2) stirring for reaction after adding dropwise of a catalyst; and
step (3) after the reaction, adding a hydrochloric acid solution, separating an organic phase after stirring and extracting the organic phase with an extractant, carrying out a rotary evaporation to obtain a crude product after drying, and purifying the crude product by a column chromatography to obtain a target product curcumin derivative;
wherein the raw material a in the step (1) has the following structural formula II: 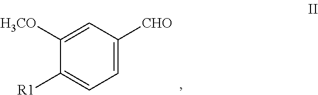
and the raw material b has the following structural formula III: 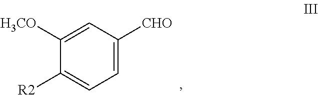
wherein R1 is one of F, Cl, Br, and I; and R2 is one of F, Cl, Br, I, and OH.

3. The method for preparing the halogenated curcumin derivative according to claim 2, wherein a molar ratio of acetylacetone, boric anhydride, the raw material a, the raw material b, and tributyl borate in the step (1) is 1:(1 to 4):(0.5 to 1.5):(0.5 to 1.5):(1 to 3); and the solvent described in the step (1) is ethyl acetate.

4. The method for preparing the halogenated curcumin derivative according to claim 2, wherein the catalyst in the step (2) is piperidine or n-butylamine, a catalyst mass is 0.1 to 1% of acetylacetone, and a stirring reaction time is 12 h to 24 h.

5. The method for preparing the halogenated curcumin derivative according to claim 2, wherein the step (1) and the step (2) are carried out under a nitrogen protection condition at 25° C. to 45° C.

6. The method for preparing the halogenated curcumin derivative according to claim 2, wherein in the step (3), a volume concentration of the hydrochloric acid solution is 10% to 30%; and a volume ratio of the hydrochloric acid solution to a solution obtained in the step (1) is (0.8 to 1.2):2.

7. An application of the halogenated curcumin derivative according to claim 1 in a preservation of an aquatic product, wherein the halogenated curcumin derivative is used as a photosensitizer for a photodynamic sterilization of the aquatic product.

8. The application of the halogenated curcumin derivative in the preservation of the aquatic product according to claim 7, wherein the photodynamic sterilization comprises the following steps:
step (A) preparing a halogenated curcumin derivative aqueous solution in a dark water tank;
step (B) draining surface water after cleaning the aquatic product with sterile normal saline;
step (C) soaking the aquatic product prepared in the step (B) in the halogenated curcumin derivative aqueous solution prepared in the step (A) by adopting a soaking method, and taking out the aquatic product after soaking for 10 min; and
step (D) immediately placing the aquatic product obtained in the step (C) under a blue LED matrix for irradiation.

9. The application of the halogenated curcumin derivative in the preservation of the aquatic product according to claim 8, wherein a concentration of the halogenated curcumin derivative aqueous solution in the step (A) is 10 mol/L to 40 mol/L.

10. The application of the halogenated curcumin derivative in the preservation of the aquatic product according to claim 8, wherein an optical power of the blue LED matrix in the step (D) is 5 W to 20 W, a wavelength range is 400 nm to 480 nm, and an irradiation time is 60 s to 180 s.
